(19) United States
(12) Patent Application Publication
(10) Pub. No.: US 2003/0050668 A1
(43) Pub. Date: Mar. 13, 2003

(54) Suture sleeve

(76) Inventor: Jonathan Lee, St. Augustine, FL (US)

Correspondence Address: Beck & Tysver, P.L.L.C., Suite 100, 2900 Thomas Avenue S., Minneapolis, MN, 55416, US

(21) Appl. No.: 10/236,620

(22) Filed: Sep. 6, 2002

Related U.S. Application Data
(60) Provisional application No. 60/317,831, filed on Sep. 7, 2001.

(51) Int. Cl. A61B 17/04

(52) U.S. Cl. 606/232

(57) ABSTRACT

A suture sleeve having a helical cut for anchoring a medical device in a body lumen.

### CROSS REFERENCE

The present application is based upon and claims the benefit of provisional application 60/317,831, which is incorporated by reference herein.

### FIELD OF INVENTION

The present invention relates generally to anchoring medical devices in the body and more particularly to a suture sleeve for securing and positioning an endovascular lead within a vessel.

### BACKGROUND OF THE INVENTION

Pacemakers and other implanted medical devices typically deliver therapeutic energy through a lead system which is implanted in an organ of the patient. In the case of a pacemaker an endovascular lead goes from the pacemaker connector block into a major blood vessel communicating with the right ventricle. The distal tip of the lead system makes contact with the wall of the heart and over time an inflammatory reaction takes place which connects the lead system to the heart wall with scar tissue. The amount of time that it takes the pacemaker lead to move from the acute to the chronic phase and be firmly attached to the wall varies depending upon the patient's immuological response to the lead as well as other variables. To prevent movement of the distal tip away from the heart wall it is conventional practice to tie a suture around the vessel where the lead enters the vessel. Typically a suture sleeve is applied to the lead to ensure that the suture does not enter the insulation of the lead. Although sutures and suture sleeves are widely used it is well documented that there can be significant motion of the lead with respect to the vessel prior to the time that the lead becomes “chronic”. Although this small migration of the lead body does not typically result in clinical problems with conventional leads, the more modern stimulating devices require more accurate positioning of the electrode system to achieve their therapeutic results. As a consequence there is a continuing need to improve the devices used to secure leads within vessels.

Conventional suture sleeves consist of a tubular structure with a longitudinal slit. This over tube is placed on the lead at the location of the entry point into the vessel and a suture may be tightly wrapped around the suture sleeve to ensure that the sleeve does not move with respect to the vessel. The problem is that the lead within the tube is restrained only by frictional forces between the sleeve and the lead body. If a tugging force is supplied to the sleeve, it will neck down very slightly reducing the frictional engagement and slip. Once it has stopped moving full frictional engagement occurs.

### SUMMARY OF THE INVENTION

The suture sleeve in accordance with the present invention includes a tubular member with a helical cut extending from a distal end to a proximal end. In operation, tensile force applied to the lead within the suture sleeve will result in an elongation of the sleeve which responds by reducing its internal diameter thereby increasing the drag or anchoring force on the lead.

Both demountable and non-mountable versions are disclosed. Although the invention is disclosed in the context of a lead placement the device can be used to anchor other medical devices in other vessels or body structures.

### BRIEF DESCRIPTION OF DRAWINGS

Throughout the several views of the drawings identical reference numerals indicate equivalent structure wherein:

FIG. 1 is a longitudinal view of a demountable suture sleeve in accordance with the invention.

FIG. 2 is a transverse view of a demountable suture sleeve in accordance with the invention.

FIG. 3 is a longitudinal view of a not removable suture sleeve in accordance with the invention.

FIG. 4 is a transverse view of a not demountable suture sleeve in accordance with the invention.

### DETAILED DESCRIPTION OF THE PREFERRED EMBODIMENTS

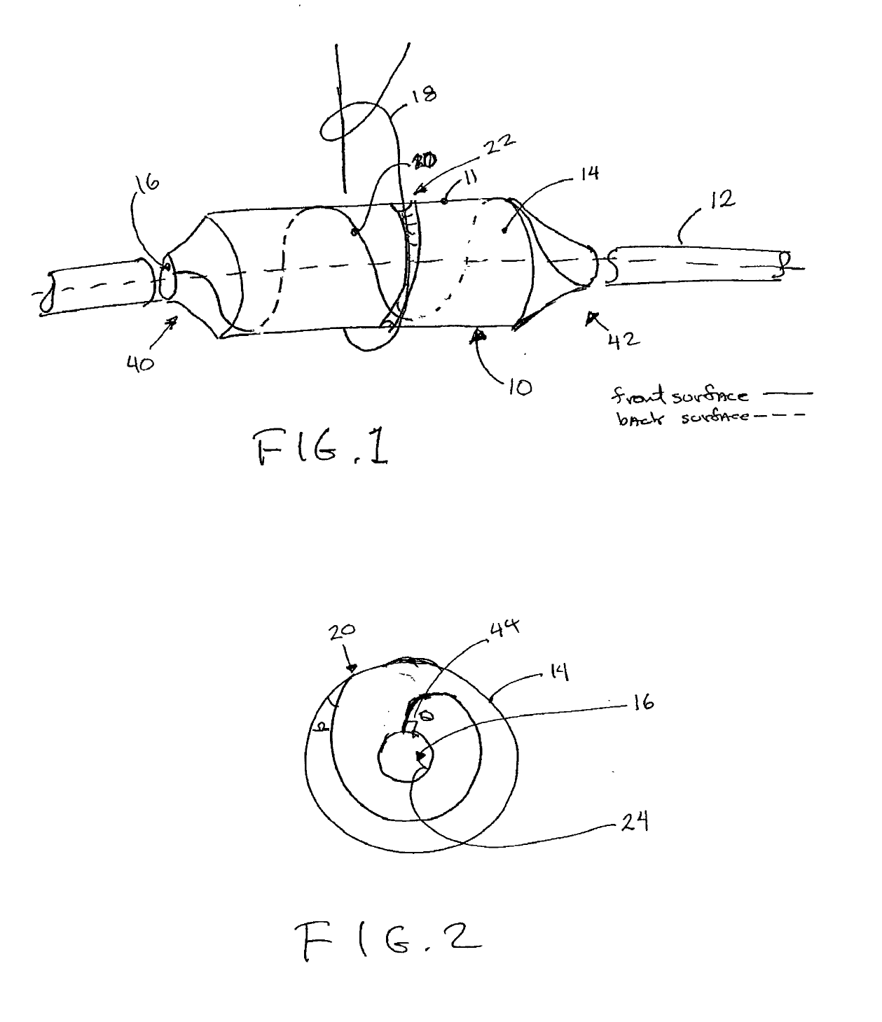

FIG. 1 shows a suture sleeve in accordance with the invention. Here a helical cut 20 is formed on the substantially cylindrical body and it extends from the substantially conical distal end 40 to the substantially conical proximal end 42 of the elongate body 11 of the device 10. This cut 20 communicates from the exterior surface 14 of the device to the interior surface 24 of the inner lumen 16 of the device. When a slight traction is applied to the device by the motion of the lead body 12 then the average interior diameter is reduced by this translational motion. It is this process in the presence of a lead body 12 which results in superior gripping and retention of the lead 12 in the suture sleeve.

It is preferred to include a suture groove 22 around the exterior surface of the device 10 to allow a centralized suture 18 to bind the device 10 and the lead body 12 together. In this construction a tensile force applied in either the proximal or distal direction each is restrained by at least one-half of the suture body.

FIG. 2 shows a transverse view of the device 10. The cut 20 forms an angle with the surface 14 which is labeled Omega or 20 in the figure This angle may be between about 90 degrees and 0 degrees, with angles near 90 degrees preferred. The cut also intersects wit the lumen 16. This angle is labeled Theta or 44 in the drawing. This angle may vary as well with the range of 0-90 degrees. The pitch of the helix is shown as constant over the length of the device for clarity however the pitch may vary continuously along the length of the device. It is expected that a coarse pitch of one turn over the length is an operational value, while fine pitch of 10 or more turns over the length of the device may be preferable in some applications. It may be preferable to have a fine pitch on the conical ends of the device while the cylindrical body may have a courser pitch.

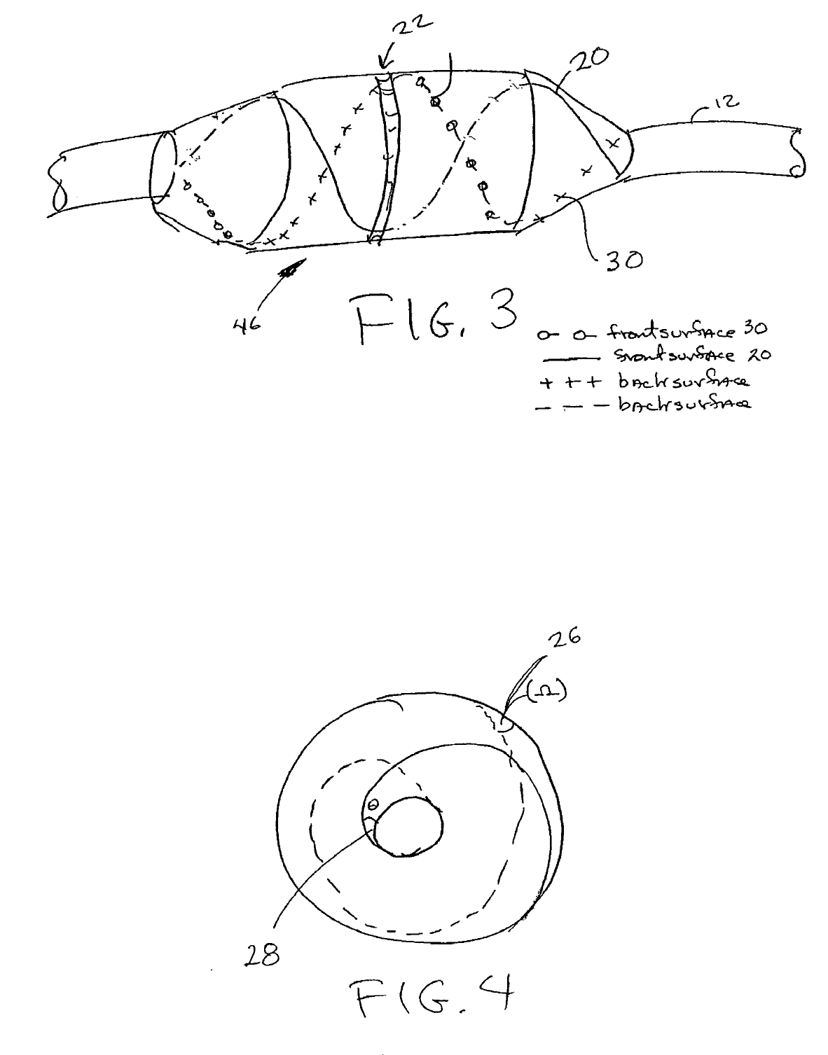

FIG. 3 shows a dual helical device with both right-handed 20 and left handed 30 cut helices. In such a device if the cuts are not completely through the to the inner lumen then the device cannot be unwound from the lead body and therefore it is a non-demountable structure. The non-demountable device must be applied to the lead body prior to introduction to the body. In this embodiment one cut is molded as a groove which does not reach the interior lumen. In this embodiment there is one helical cut and an opposed helical groove rendering the device demountable. In summary, a device with a cut is demountable and a completely grooved embodiment is not demountable. It should be apparent that grooved and cut embodiments are possible and devices with an interrupted cut may be constructed as well.

FIG. 4 shows a transverse cross section of the device of FIG. 3. In this figure the right hand cut 20 is shown in solid outline while the dotted spiral reflects the left-hand spiral or helical cut 30.

What is claimed is:

1. A suture sleeve device for anchoring a medical device in a body lumen comprising:
an approximately cylindrical elongate body having a distal end and a proximal end and having an exterior surface;
a lumen extending the length of said elongate body lying along an axis;
a helical cut piercing said body extending from said lumen to said exterior surface;
said cut forming a first angle with respect to said lumen and a second angle to said exterior surface said helical cut extending completely through said body.

2. The device of claim 1 wherein said helical cut has a fine pitch.

3. The device of claim 1 wherein said helical cut has a coarse pitch.

4. The device of claim 1 wherein said helical cut is right handed.

5. The device of claim 1 wherein said helical cut is left handed.

6. The device of claim 1 wherein said lumen is substantially cylindrical.

7. The device of claim 1 wherein said device has a center cylindrical body and a tapered distal end and a tapered proximal end.

8. The device of claim 1 wherein the device has a left handed helical cut on f said distal end or said proximal end, and a complimentary right handed helical cut on said other of said distal and proximal ends; the cuts arranged to not intersect.

9. A suture sleeve device for anchoring a medical device in a body lumen comprising:
an approximately cylindrical elongate body having a distal end and a proximal end and having an exterior surface;
a lumen extending the length of said elongate body lying along an axis;
a first helical cut piercing said body extending from said lumen to said exterior surface;
said first helical cut extending completely through said elongate body in a left hand direction;
a second helical cut piercing said body extending from said lumen to said exterior surface;
said second helical cut extending partially through said elongate body wound in a right hand direction.
